(19) United States
(12) Patent Application Publication
(10) Pub. No.: US 2007/0099186 A1
(43) Pub. Date: May 3, 2007

(54) Methods and means for the treatment of disorders associated with cellular senescence

(76) Inventors: Fabrizio D'Adda Di Fagagna, Cambridge (GB); Philip Michael Reaper, Cambridge (GB); Stephen Jackson, Cambridge (GB)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/554,878

(22) PCT Filed: Apr. 21, 2004
(86) PCT No.: PCT/GB04/01712

§ 371 Date: Sep. 1, 2006

Related U.S. Application Data
(60) Provisional application No. 60/467,395, filed on May 1, 2003.

(51) Int. Cl. C12Q 1/68; G01N 33/567; C12Q 1/48; A61K 31/55; A61K 31/4433; A61K 31/4436; A61K 31/453; A61K 31/452; A61K 31/4025; A61K 31/397

(52) U.S. Cl. 435/6; 435/7.2; 435/15; 514/460; 514/336; 514/210.19; 514/217.03; 514/326; 514/422

(57) ABSTRACT

This invention relates to mechanisms of cellular senescence and, in particular, to the role of DNA repair and DNA damage checkpoint pathways in the induction and maintenance of the senescent state. Methods and means of inducing cell cycle progression in senescent cells by inhibiting DNA damage checkpoint pathways are provided herein. These methods and means provide agents and therapies for the treatment of senescent associated disorders.

This invention relates to mechanisms of cellular senescence and, in particular, to the role of DNA repair and DNA damage checkpoint pathways in the induction and maintenance of the senescent state.

Somatic human cells generally divide for only a limited number of population doublings in vitro under standard tissue culture conditions. When cell proliferation in a culture spontaneously ceases, most cells acquire an enlarged morphology and express a range of markers, some of which are also associated with cellular stress. Although senescent cells are unable to divide, they are nevertheless metabolically active and can be maintained in culture for long periods of time. Sub-optimal growth conditions can be responsible for the growth arrest and senescent phenotypes have been observed with some cultured rodent cells (Tang, D.G. et al. Science 291, 868-71 (2001), Mathon, N.F. et al. Science 291, 872-5 (2001)). However, under standard culture conditions, the life span of primary human diploid fibroblasts (HDFs) from healthy donors depends on the state of their telomeres.

In most somatic human cells, the ends of linear chromosomes—telomeres—are not fully replicated before each cell division, and as a consequence successive generations of daughter cells inherit chromosomes with progressively eroded telomeres. In some HDF lines, the ectopic expression of the catalytic component of telomerase (hTERT)—an enzyme able to elongate telomeres—counteracts progressive telomere attrition and confers an apparently infinite extension on the culture lifespan (Bodnar, A. G. et al. Science 279, 349-352 (1998)). Thus, the arrest of the cell cycle and the induction of senescence in HDFs are primarily governed by telomere shortening.

The present inventors have recognised that responses similar to those induced by DNA double strand breaks (DSBS) play an active role in inducing and maintaining cell cycle arrest in senescent cells. Furthermore, inhibition of the DNA damage checkpoint pathway is shown herein to cause reactivation of the cell cycle in senescent cells.

DNA DSBs are the primary cytotoxic lesions caused by ionising radiation (IR) and radio-mimetic drugs. Cells react to DSBs by mounting a range of responses, including the activation of DNA repair mechanisms and the triggering of checkpoint events whose primary function is to halt or slow cell cycle progression until the DNA damage has been removed (Shiloh, Y. Nature Reviews Cancer 3, 155-68 (2003), Nyberg, K. A. et al Annu Rev Genet 36, 617-56 (2002), Khanna & Jackson Nat. Genet 27 247-254 (2001)). Treatment of human cells with IR leads to the rapid activation of the DNA-damage transducer protein kinases ATM and ATR. These kinases then phosphorylate and activate a series of downstream targets, including the effector protein kinases CHK1 and CHK2, and the checkpoint mediator proteins 53BP1 and MDC1. In addition, ATM and ATR phosphorylate the histone variant H2AX on Ser-139; this response can be detected within a minute of IR exposure and eventually extends over a large domain of chromatin flanking the site of DNA damage. This evolutionarily conserved response can be triggered by as little as one DNA DSB (Chen, H. T. et al. Science 290, 1962-1964 (2000)) and is widely recognised as a specific and unequivocal marker for the in vivo generation of this type of damage. The phosphorylation of histone H2AX then facilitates the recruitment to sites of DNA damage of a series of checkpoint and DNA repair factors, including 53BP1, MDC1, the MRE11/RAD50/NBS1 complex and the phosphorylated form of the structural maintenance of chromosomes 1 (SMCl) protein. The formation of these foci at sites of DNA DSBs is characteristic feature of the checkpoint response (Goldberg, M. et al. Nature 421, 952-6 (2003)).

Various aspects of the invention relate to the identification of agents which inhibit the DNA damage checkpoint response pathway and which may be used to initiate cell cycle progression in senescent cells, for example in the treatment of a disorder associated with senescence.

One aspect of the invention provides a method of identifying an agent for the treatment of a senescence associated disorder comprising:
contacting a test compound with a DNA damage checkpoint response polypeptide;
determining binding of the polypeptide by the test compound.

Binding of the DNA damage checkpoint pathway polypeptide by the test compound is indicative that the test compound has an inhibitory effect on the polypeptide and is a candidate agent for the treatment of senescence associated disorders.

Senescence associated disorders include any disorder which is fully or partially mediated by the induction or maintenance of a non-proliferating or senescent state in a cell or a population of cells in an individual. Examples include coronary disease, impaired wound healing, immune dysfunction, age-related tissue or organ decline, Alzheimer's disease, liver cirrhosis and immuno-senescence caused by chronic infection by agents such as HIV.

A DNA damage checkpoint response polypeptide is a polypeptide which is active in mediating the activation of a cell cycle checkpoint in response to DNA damage, in particular double strand breaks i.e. a polypeptide which is component of the DNA damage checkpoint response pathway. Preferably, the polypeptide plays little or no role in other cellular signalling pathways. Suitable polypeptides include ATM, ATR, ATRIP, CHK1, CHK2, BRCA1, NBS1, RAD50, MRE11, CDC25C, 14-3-3σ, CDK2/cyclin E, CDK2/cyclin B1 53BP1, MDC1, histone variant H2AX, SMC1, RAD17, RAD1, RAD9, HUS1 and MRC1. The DNA damage checkpoint response as described herein includes both ATM and ATR dependent signalling pathways and is described in more detail in Khanna and Jackson (2001) supra.

The nucleic acid and protein sequences of various components of the DNA damage checkpoint pathway in humans and yeast are available from the GenBank database, under the following accession numbers: Human ATM (Nucleic acid coding sequence (CDS): W82828, protein sequence: AAB65827, Human CHK1 (CDS: AF016582, protein: AAC51736), Human CHK2 (CDS: NM_{—}007194, protein: 096017), NBS1 (CDS: AF3169124, protein: BAA28616), Human RAD50 (CDS: 5032016, protein: NP_{—}005723), MRE11 (CDS: U37359, protein: AAC78721), BRCA1 (CDS: U14680, protein: A58881), ATR, (CDS: NM_{—}001184, protein: NP_{—}001175) ATRIP (CDS: AF451323, protein: AAL38042.1), CDC25C (CDS: NM_{—}001790, protein: NP 001781.1), 53BP1 (CDS: NM_{—}005657, protein: NP_{—}005648), MDC1 (CDS: NM_{—}014641 protein: NP_{—}055456), histone variant H2AX (CDS: NM_{—}002105, protein: NP_{—}002096), SMC1 (CDS: NM_{—}006306, protein: NP_{—}006297), RAD17 (CDS: NM_{—}133338, protein: NP_{—}579916), RAD1 (CDS: NM_{—}002853, protein: NP_{—}002844), RAD9 (CDS: NM_{—}004584, protein: NP_{—}004575), HUS1 (CDS: NM_{—}148959, protein: NP_{—}683762) and MRC1 (CDS: NM_{—}002438, protein: NP_{—}002429).

In other embodiments, a method of identifying an agent for the treatment of a senescence-associated disorder may comprise:
contacting a DNA damage checkpoint response polypeptide with a substrate molecule in the presence of a test compound;
under conditions in which the DNA damage checkpoint response polypeptide normally interacts with the substrate molecule; and,
determining interaction of the polypeptide and the substrate molecule,
a decrease in said interaction in the presence relative to the absence test compound being indicative that the test compound is a candidate agent for the treatment of senescence associated disorders.

Interaction may be determined for example by determining binding between the polypeptide and the substrate molcule.

A substrate molecule may be any molecule which normally interacts with the DNA damage checkpoint response polypeptide. Substrates for DNA damage checkpoint response polypeptides may include polypeptides, for example other DNA damage checkpoint response polypeptides or fragments or derivatives thereof. A polypeptide substrate may, for example, be phosphorylated by a DNA checkpoint kinase. Other substrates may be nucleic acid molecules. A nucleic acid substrate may, for example, be cleaved by al DNA checkpoint nuclease, or topologically rearranged by a DNA checkpoint helicase or bound by a DNA checkpoint binding factor. DNA damage response checkpoint polypeptides and substrate molecules are described in Khanna & Jackson (2001) supra.

The determination of the interaction of a DNA damage checkpoint response polypeptide and substrate molecule by determining the enzymatic modification, for example phosphorylation, of the substrate molecule is discussed in more detail below.

It is not necessary to use the entire full-length proteins for methods of the invention, whether in vitro or in vivo. Polypeptide fragments which retain all or part of the activity of the full-length protein may be generated and used in any suitable way known to those of skill in the art. Suitable ways of generating fragments include, but are not limited to, recombinant expression of a fragment from encoding DNA. For example, fragments may be generated by taking encoding DNA, identifying suitable restriction enzyme recognition sites either side of the portion to be expressed, and cutting out said portion from the DNA. The portion may then be operably linked to a suitable promoter in a standard commercially available expression system. Another recombinant approach is to amplify the relevant portion of the DNA with suitable PCR primers. Small fragments (e.g. up to about 20 or 30 amino acids) may also be generated using peptide synthesis methods which are well known in the art as further described below.

Of course, reference to a polypeptide or protein of the DNA damage checkpoint pathway may be taken to refer to a derivative, variant or analogue of the relevant polypeptide or protein which has the requisite, assayable property or activity (e.g. ability to bind, activate or be activated by another component in the pathway).

Methods according to the present invention may be in vivo cell-based methods, or in vitro non-cell-based methods. The precise format for performing methods of the invention may be varied by those of skill in the art using routine skill and knowledge.

Methodologies for identifying or obtaining compounds which modulate the interaction between two molecules are well-known in the art and include techniques such as radioimmunosassay, scintillation proximetry assay and ELISA methods.

For example, interaction between components of the DNA damage checkpoint response pathway and their substrates may be studied in vitro by labelling one with a detectable label and bringing it into contact with the other which has been immobilised on a solid support. This may be performed in the presence of a test compound.

Suitable detectable labels, especially for peptidyl substances include ³⁵S-methionine, which may be incorporated into recombinantly produced peptides and polypeptides. Recombinantly produced peptides and polypeptides may also be expressed as a fusion protein containing an epitope which can be labelled with an antibody.

In a scintillation proximetry assay, a biotinylated protein fragment may be bound to streptavidin coated scintillant-impregnated beads (for example, produced by Amersham). Binding of radiolabelled peptide is then measured by determination of radioactivity-induced scintillation as the radioactive peptide binds to the immobilized fragment. Agents which intercept this are inhibitors of the interaction.

A polypeptide may be immobilized using an antibody against that polypeptide which is bound to a solid support or via other technologies which are known per se. A preferred in vitro interaction may utilise a fusion protein including glutathione-S-transferase (GST). This may be immobilized on glutathione agarose beads. In an in vitro format, a test compound can be assayed by determining its ability to diminish the amount of labelled peptide or polypeptide which binds to the immobilized GST-fusion polypeptide. This may be determined by fractionating the glutathione-agarose beads by SDS-polyacrylamide gel electrophoresis.

Alternatively, the beads may be rinsed to remove unbound protein and the amount of bound protein determined by counting the amount of label present, for example, using a suitable scintillation counter.

Of course, the person skilled in the art will design any appropriate control experiments with which to compare results obtained in methods of the invention.

Methods of the invention may also take the form of in vivo methods. In vivo methods may be performed in a cell line such as a yeast strain, insect or mammalian cell line, for example CHO, HeLa or COS cells, in which the relevant polypeptides or peptides are expressed from one or more vectors introduced into the cell.

Other suitable techniques include the yeast two-hybrid system (e.g Evan et al. Mol. Cell. Biol. 5, 3610-3616 (1985); Fields & Song Nature 340, 245-246 (1989)). This system often utilises a yeast containing a GAL4 responsive promoter linked to β-galactosidase gene and to a gene (HIS3) that allows the yeast to grow in the absence of the amino acid histidine and to grow in the presence of the toxic compound 3-aminotriazole. The pathway polypeptide may be cloned into a yeast vector that will express the protein as a fusion with the DNA binding domain of GAL4. The yeast may then be transformed with DNA libraries designed to express test polypeptides or peptides as GAL4 activator fusions. Yeast that have a blue colour on indicator plates (due to activation of β-galactosidase) and will grow in the absence of histidine (and the presence of 3-aminotriazole) may be selected and the library plasmid isolated. The library plasmid may encode a compound that can interact with the DNA damage checkpoint pathway polypeptide.

A variation on this may be used to screen for compounds able to disrupt interaction between two polypeptides which are components of the DNA damage checkpoint pathway. For instance, the components may be expressed in a yeast two-hybrid system (e.g. one as a GAL4 DNA binding domain fusion, the other as a GAL4 activator fusion) which is treated with test substances. The absence of the end-point which normally indicates interaction between the pathway components (e.g. the absence of a blue colour in the exemplary system outlined above) when a test compound is applied indicates that compound disrupts interaction between the two components, and may therefore inhibit the DNA damage checkpoint pathway, indicative of potential as an inducer of cell cycle progression in a senescent cell.

A method of identifying an agent for the treatment of a senescence associated disorder as described herein may include determining the activity of a DNA damage checkpoint pathway polypeptide, for example, in the presence and absence of said test compound.

For example, a method of identifying an agent for the treatment of a senescence associated disorder may comprise:
contacting a DNA damage checkpoint response polypeptide with a test compound; and,
determining the activity of the polypeptide.

A decrease in the activity of the DNA damage checkpoint pathway polypeptide in the presence relative to the absence of test compound is indicative that the test compound is a candidate agent for the treatment of senescence-associated disorders.

Activities which may be determined may include kinase, helicase, nuclease, and ribonucleotide reductase (RNRase). These activities may be determined using conventional techniques. In some preferred embodiments, for example when the polypeptide is ATM, ATR, Chk1 or Chk2, the kinase activity of the polypeptide may be determined.

In some embodiments, activity may be determined by contacting the DNA damage checkpoint response polypeptide with the test compound in the presence of a substrate of said polypeptide. Activity of the DNA damage checkpoint response polypeptide may be determined by determining the depletion of unmodified substrate or the formation of product (i.e. substrate modified by the polypeptide). Suitable substrate molecules are discussed above and may include natural or artificial substrates of a DNA damage checkpoint response polypeptide. An artificial substrate may be a derivative or analogue of a natural substrate of the polypeptide.

The phosphorylation of a DNA damage checkpoint pathway polypeptide may be indicative of its activated state. Activity may also therefore be determined by determining the phosphorylation of a DNA damage checkpoint pathway polypeptide. DNA damage checkpoint pathway polypeptides which are activated by phosphorylation include ATRIP, CHK1, CHK2, BRCA1, NBS1, RAD50, MRE11, CDC25C, 14-3-3σ, CDK2/cyclin E, CDK2/cyclin B1 53BP1, MDC1, histone variant H2AX, SMC1, RAD17, RAD1, RAD9, HUS1 and MRC1.

In some embodiments, methods of the invention may comprise determining the effect of a test compound on all or part of the DNA damage checkpoint pathway.

A method of screening for an agent for the treatment of a senescence associated disorder, may comprise:
providing a DNA damage checkpoint pathway;
exposing the pathway to a test compound under conditions which would normally lead to the activation of the DNA damage pathway; and,
determining the activation of the DNA damage checkpoint pathway in the presence relative to the absence of test compound.

The pathway may be provided in a cell to be exposed to the test compound. Suitable cells include eukaryotic cells such as yeast, amphibian, avian or mammalian cells. Numerous cultured cell lines which possess a suitable DNA damage checkpoint pathway are available.

Activation of the DNA damage checkpoint pathway may be determined by any convenient method. For example, activation of the pathway may be determined by determining the activation of one or more components of the pathway. In some embodiments, for example, the kinase activity of one or more DNA damage checkpoint kinases, such as ATM, ATR, CHK1 or CHK2 may be determined.

In other embodiments, activation of the DNA damage checkpoint pathway may be determined by determining the phosphorylation of one or more DNA damage checkpoint polypeptides. Examples of DNA damage checkpoint polypeptides which are activated by phosphorylation include ATRIP, CHK1, CHK, BRCA1, NBS1, RAD50, MRE11, CDC25C, 14-3-3σ, CDK2/cyclin E, CDK2/cyclin B1 53BP1, MDC1, histone variant H2AX, SMC1, RAD17, RAD1, RAD9, HUS1 and MRC1.

Phosphorylation may be determined by any suitable method known to those skilled in the art. It may be detected by methods employing radiolabelled ATP and optionally a scintillant. By way of example, phosphorylation of a protein may be detected by capturing it on a solid substrate using an antibody or other specific binding molecule directed against the protein and immobilised to the substrate, the substrate being impregnated with a scintillant—such as in a standard scintillation proximity assay. Phosphorylation is then determined via measurement of the incorporation of radioactive phosphate.

Phosphate incorporation may also be determined by precipitation with acid, such as trichloroacetic acid, and collection of the precipitate on a nitrocellulose filter paper, followed by measurement of incorporation of radiolabelled phosphate.

Phosphorylation may also be detected by methods employing an antibody or other binding molecule which binds the phosphorylated polypeptide with a different affinity to unphosphorylated polypeptide. Such antibodies may be obtained by means of any standard technique as discussed elsewhere herein. Binding of a binding molecule which discriminates between the phosphorylated and non-phosphorylated form of a polypeptide may be assessed using any technique available to those skilled in the art, examples of which are discussed elsewhere herein.

In some preferred embodiments of the invention, DNA damage checkpoint pathway polypeptides may exclude p53, p53R1 and p21 and other polypeptides known to mediate other cellular signalling pathways.

As other end points for screens, the effect on the repair of DNA damage, or cell viability or proliferation may be measured. Suitable methods are known to those skilled in the art.

The inhibitory activity of a compound on the DNA damage checkpoint pathway may be verified by one or more of the following; hypersensitivity of mammalian cells to ionising radiation, by rejoining of double-strand breaks (e.g. in a plasmid or in chromosomal DNA) in vivo (e.g. a Comet assay: Schindewolf 2000 Mammalian Genome 11 552-554), defects in the slowing or arrest of cell cycle progression following DNA damage, defects in the slowing or arrest of entry into the apoptotic program in response to DNA damage or defects in the phosphorylation or other modification of proteins in response to DNA damage.

Activation of the DNA damage checkpoint pathway may also be determined by determining the presence of nuclear foci of one or more DNA damage checkpoint polypeptides. Foci formed by DNA repair and DNA damage signalling proteins are termed SAFs (senescence associated foci) and are shown herein to be diagnostic for senescent cells. DNA damage checkpoint polypeptides which form foci in the nuclei of senescent cells include γH2AX (C terminally phosphorylated form of Histone H2AX), 53BP1, MDC1, NBS1, RAD50, MRE11, SMC1, and RAD51. Foci may be detected, for example, by standard immunofluorescent techniques, as described herein.

The inhibition of the DNA damage checkpoint response is shown herein to induce cell division in senescent (i.e. ‘non-proliferating’) cells. Methods of the invention may comprise determining the ability of a test compound to induce or stimulate the progression of the cell cycle in a senescent cell. Cell cycle progression may be determined by any convenient technique including monitoring the incorporation of BrdU.

Appropriate control experiments may be performed in accordance with appropriate knowledge and practice of the ordinary skilled person. Experiments may, for example, be performed in the presence and absence of a test compound. A decrease in activity or activation in the presence, relative to the absence of the compound may be indicative that the compound has an inhibitory effect on the pathway or a component thereof.

The DNA damage checkpoint pathway or polypeptide may be human, non-human mammalian or avian, bearing in mind veterinary applications. However, given the ease of manipulation of lower eukaryotes, such as yeast, and the good conservation between DNA damage checkpoint polypeptides in different eukaryotes, methods of the invention may conveniently involve applying test substances to a yeast system with the expectation that similar results will be obtained using the substances in mammalian, e.g. human, systems. In other words, a compound identified as being able to inhibit the DNA damage checkpoint pathways in yeast may also be able to inhibit the DNA damage checkpoint pathway in other eukaryotes. A further approach, as discussed, is to use yeast cells expressing one or more components (e.g. ATM, ATR, CHK1 or CHK2) of the DNA damage checkpoint pathway of another eukaryote, e.g. human. In some embodiments, a plant DNA damage checkpoint pathway or one or more polypeptide components thereof may be employed to test for substance useful in reversing cell cycle arrest and inducing proliferation in senescent cells.

Alternatively, methods may be performed on an in vitro DNA damage checkpoint system that measures the phosphorylation of substrates or the accuracy and efficiency of joining together DNA strand breaks that have been created by treating intact DNA with restriction endonucleases, chemicals, or radiation.

Test compounds for use in methods of the invention may be natural or synthetic chemical compounds used in drug screening programmes. Extracts of plants which contain several characterised or uncharacterised components may also be used. Combinatorial library technology (Schultz, JS (1996) Biotechnol. Prog. 12:729-743) provides an efficient way of testing a potentially vast number of different substances for ability to modulate activity of a polypeptide.

The amount of test substance or compound which may be added to a method of the invention will normally be determined by trial and error depending upon the type of compound used. Typically, from about 0.1 to 100 μM concentrations of putative inhibitor compound may be used, for example from 1 to 10 μM. When cell-based methods are employed, the test substance or compound is desirably membrane permeable in order to access the interacting polypeptides.

One class of putative agents for inducing cell cycle progression in senescent cells can be derived from the DNA damage checkpoint response polypeptides as described above. Membrane permeable peptide fragments of from 5 to 40 amino acids, for example, from 6 to 10 amino acids may be tested for their ability to disrupt the DNA damage checkpoint response.

The inhibitory properties of a peptide fragment as described above may be increased by the addition of one of the following groups to the C terminal: chloromethyl ketone, aldehyde and boronic acid. These groups are transition state analogues for serine, cysteine and threonine proteases. The N terminus of a peptide fragment may be blocked with carbobenzyl to inhibit aminopeptidases and improve stability (Proteolytic Enzymes 2nd Ed, Edited by R. Beynon and J. Bond, Oxford University Press, 2001).

Other putative agents for the induction of cell cycle progression in senescent cells include known inhibitors of the DNA damage checkpoint response. Suitable ATM inhibitors, for example are described in PCT/GB03/000770 and include compounds having the formula: 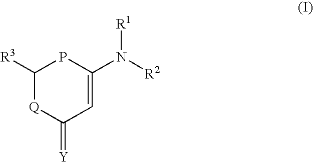
and isomers, salts, solvates, chemically protected forms, and prodrugs thereof, wherein:

one of P and Q is O, and the other of P and Q is CH, where there is a double bond between whichever of Q and P is CH and the carbon atom bearing the R³ group;

Y is either O or S, preferably O;

R¹ and R² are independently hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;

R³ is a phenyl or pyridyl group, preferably a phenyl group, attached by a first bridge group selected from —S—, —S(═O)—,—S(═O)₂—, —O—, —NR^{N}— and CR^{C1}RC²— to an optionally substituted C₅₋₂₀ carboaryl group, in which one aromatic ring atom may be replaced by a nitrogen ring atom;

the phenyl or pyridyl group and optionally substituted C₅₋₂₀ carboaryl group being optionally further linked by a second bridge group, which is bound adjacent the first bridge group on both groups so as to form an optionally substituted C₅₋₇ ring fused to both the phenyl or pyridyl group and the C₅₋₂₀ carboaryl group, the phenyl or pyridyl group being further optionally substituted;

wherein R^{N} is selected from hydrogen, an ester group, an optionally substituted C₁₋₇ alkyl group, an optionally substituted C₃₋₂₀ heterocyclyl group and an optionally substituted C₅₋₂₀ aryl group;

and R^{C1} and R^{C2} are independently selected from hydrogen, an optionally substituted C₁₋₇ alkyl group, an optionally substituted C₃₋₂₀ heterocyclyl group and an optionally substituted C₅₋₂₀ aryl group.

For example, a compound may have the formula Ia:

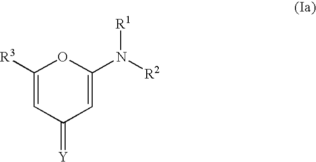

In some preferred embodiments, R¹ and R² form, along with the nitrogen atom to which they are attached, a heterocyclic ring having 6 ring atoms, for example a morpholino or thiomorpholino group.

The phenyl or pyridyl ring or the C₅₋₂₀ carboaryl group in R³ may bear a substituent selected from the group consisting of acylamido, sulfonamino, ether, ester, amido and acyl.

R³ may for example be selected from the following optionally substituted groups

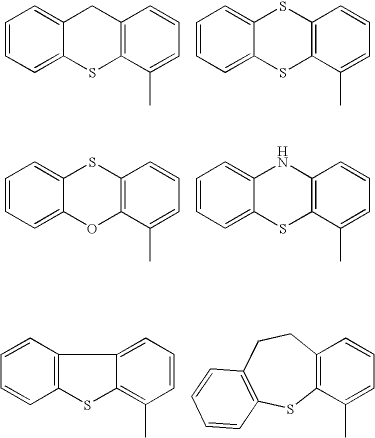

Antibodies directed to the active site or site of interaction of a DNA damage checkpoint response polypeptide form a further class of putative agents for inducing cell cycle progression in senescent cells. Candidate inhibitor antibodies may be characterised and their binding regions determined to provide single chain antibodies and fragments thereof which are responsible for disrupting the interaction. Antibodies may also be useful in determining the presence of activated DNA damage checkpoint response polypeptides in a cell, for example in methods of identifying a senescent cell. Suitable antibodies may, for example, be specific for the active phosphorylated form of a polypeptide, such that the binding of the antibody is indicative of the presence of an activated DNA damage checkpoint response polypeptide in a cell. Antibodies may be useful in the detection of senescent cells, for example in the study and diagnosis of disease conditions and the evaluation of potential therapies.

Antibodies may be obtained using techniques which are standard in the art. Methods of producing antibodies include immunising a mammal (e.g. mouse, rat, rabbit, horse, goat, sheep or monkey) with the protein or a fragment thereof. Antibodies may be obtained from immunised animals using any of a variety of techniques known in the art, and screened, preferably using binding of antibody to antigen of interest. For instance, Western blotting techniques or immunoprecipitation may be used (Armitage et al., 1992, Nature 357: 80-82). Isolation of antibodies and/or antibody-producing cells from an animal may be accompanied by a step of sacrificing the animal.

As an alternative or supplement to immunising a mammal with a peptide, an antibody specific for a protein may be obtained from a recombinantly produced library of expressed immunoglobulin variable domains, e.g. using lambda bacteriophage or filamentous bacteriophage which display functional immunoglobulin binding domains on their surfaces; for instance see WO92/01047. The library may be naive, that is constructed from sequences obtained from an organism which has not been immunised with any of the proteins (or fragments), or may be one constructed using sequences obtained from an organism which has been exposed to the antigen of interest.

Antibodies may be modified in a number of ways. Indeed, the term “antibody” should be construed as covering any binding substance having an immunoglobulin binding domain with the required specificity, including antibody fragments and derivatives.

Example antibody fragments, capable of binding an antigen or other binding partner are the Fab fragment consisting of the VL, VH, Cl and CH1 domains; the Fd fragment consisting of the VH and CH1 domains; the Fv fragment consisting of the VL and VH domains of a single arm of an antibody; the dAb fragment which consists of a VH domain; isolated CDR regions and F(ab′)2 fragments, a bivalent fragment including two Fab fragments linked by a disulphide bridge at the hinge region. Single chain Fv fragments are also included.

Antibodies may be useful in a therapeutic context (which may include prophylaxis) to disrupt interactions between with DNA damage response pathway components with a view to inhibiting the activity of the pathway and inducing or stimulating cell division. Antibodies may also be employed for other therapeutic and non-therapeutic purposes which are discussed elsewhere herein.

Other candidate compounds for inducing cell cycle progression in senescent cells may be based on modelling the 3-dimensional structure of a polypeptide component of the DNA damage checkpoint pathway and using rational drug design to provide candidate compounds with particular molecular shape, size and charge characteristics.

A candidate compound for the induction of cell cycle progression in senescent cells may be a “functional analogue” of a peptide or other compound which inhibits the DNA damage checkpoint response in a method of the invention. A functional analogue has the same functional activity as the peptide or other compound in question, i.e. it may interfere with the interactions or activity of one or more components of the DNA damage checkpoint response pathway. Examples of such analogues include chemical compounds which are modelled to resemble the three dimensional structure of the component in an area which contacts another component, and in particular the arrangement of the key amino acid residues as they appear.

Components of the DNA damage checkpoint response may be used in methods of designing mimetics of these molecules suitable for inhibiting the response and stimulating cell cycle progression and cell division in senescent cells.

Accordingly, the present invention provides a method of designing mimetics of components of the DNA damage checkpoint response having the biological activity of stimulating cell cycle progression and cell division in senescent cells; said method comprising:

(i) analysing a substance having the biological activity to determine the amino acid residues essential and important for the activity to define a pharmacophore; and,

(ii) modelling the pharmacophore to design and/or screen candidate mimetics having the biological activity.

Suitable modelling techniques are known in the art. This includes the design of so-called “mimetics” which involves the study of the functional interactions of the molecules and the design of compounds which contain functional groups arranged in such a manner that they could reproduced those interactions.

The modelling and modification of a ‘lead’ compound to optimise its properties, including the production of mimetics, is further described below.

As described above, the activity of the DNA damage response may be inhibited, as noted, by means of a substance that interferes in some way with the interaction between components of the response pathway. An alternative approach to inhibition employs regulation at the nucleic acid level to inhibit activity or function by down-regulating production of DNA damage response components.

For instance, expression of a gene may be inhibited using anti-sense or RNAi technology. The use of these approaches to down-regulate gene expression is now well-established in the art.

Anti-sense oligonucleotides may be designed to hybridise to the complementary sequence of nucleic acid, pre-mRNA or mature mRNA, interfering with the production of MRIP1 polypeptide so that its expression is reduced or completely or substantially completely prevented. In addition to targeting coding sequence, antisense techniques may be used to target control sequences of a gene, e.g. in the 5′ flanking sequence, whereby the antisense oligonucleotides can interfere with expression control sequences. The construction of antisense sequences and their use is described for example in Peyman and Ulman, Chemical Reviews, 90:543-584, (1990) and Crooke, Ann. Rev. Pharmacol. Toxicol., 32:329-376, (1992).

Oligonucleotides may be generated in vitro or ex vivo for administration or anti-sense RNA may be generated in vivo within cells in which down-regulation is desired. Thus, double-stranded DNA may be placed under the control of a promoter in a “reverse orientation” such that transcription of the anti-sense strand of the DNA yields RNA which is complementary to normal mRNA transcribed from the sense strand of the target gene. The complementary anti-sense RNA sequence is thought then to bind with mRNA to form a duplex, inhibiting translation of the endogenous mRNA from the target gene into protein. Whether or not this is the actual mode of action is still uncertain. However, it is established fact that the technique works.

The complete sequence corresponding to the coding sequence in reverse orientation need not be used. For example fragments of sufficient length may be used. It is a routine matter for the person skilled in the art to screen fragments of various sizes and from various parts of the coding or flanking sequences of a gene to optimise the level of anti-sense inhibition. It may be advantageous to include the initiating methionine ATG codon, and perhaps one or more nucleotides upstream of the initiating codon. A suitable fragment may have about 14-23 nucleotides, e.g. about 15, 16 or 17.

An alternative to anti-sense is to use a copy of all or part of the target gene inserted in sense, that is the same, orientation as the target gene, to achieve reduction in expression of the target gene by co-suppression; Angell & Baulcombe (1997) The EMBO Journal 16,12:3675-3684; and Voinnet & Baulcombe (1997) Nature 389: pg 553). Double stranded RNA (dsRNA). has been found to be even more effective in gene silencing than both sense or antisense strands alone (Fire A. et al Nature, Vol 391, (1998)). dsRNA mediated silencing is gene specific and is often termed RNA interference (RNAi).

RNA interference is a two step process. First, dsRNA is cleaved within the cell to yield short interfering RNAs (siRNAs) of about 21-23nt length with 5′ terminal phosphate and 3′ short overhangs (˜2nt). The siRNAs target the corresponding mRNA sequence specifically for destruction (Zamore P.D. Nature Structural Biology, 8, 9, 746-750, (2001)

RNAi may be also be efficiently induced using chemically synthesized siRNA duplexes of the same structure with 3′-overhang ends (Zamore PD et al Cell, 101, 25-33, (2000)). Synthetic siRNA duplexes have been shown to specifically suppress expression of endogenous and heterologeous genes in a wide range of mammalian cell lines (Elbashir SM. et al. Nature, 411, 494-498, (2001)).

Another possibility is that nucleic acid is used which on transcription produces a ribozyme, able to cut nucleic acid at a specific site—thus also.useful in influencing gene expression. Background references for ribozymes include Kashani-Sabet and Scanlon, 1995, Cancer Gene Therapy, 2(3): 213-223, and Mercola and Cohen, 1995, Cancer Gene Therapy, 2(1), 47-59.

A method as described herein may comprise the step of identifying a test compound as an agent which inhibits the DNA damage checkpoint response and which is therefore a candidate agent for inducing cell cycle progression in a senescent cell.

Following identification of a compound which inhibits the DNA damage checkpoint response, the compound may be investigated further, in particular for its ability to induce proliferation in senescent cells. A test compound may be identified as an agent which induces cell cycle progression in a senescent cell.

The test compound may be isolated and/or purified or alternatively it may be synthesised using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of senescence-associated disorders as described below. Methods of the invention may thus comprise formulating said test compound in a pharmaceutical composition with a pharmaceutically acceptable excipient, vehicle or carrier for therapeutic application, as discussed further below.

Following identification of a compound which induces cell cycle progression in a senescent cell as described above, a method may further comprise modifying the compound to optimise the pharmaceutical properties thereof.

The modification of a ‘lead’ compound identified as biologically active is a known approach to the development of pharmaceuticals and may be desirable where the active compound is difficult or expensive to synthesise or where it is unsuitable for a particular method of administration, e.g. peptides are not well suited as active agents for oral compositions as they tend to be quickly degraded by proteases in the alimentary canal. Modification of a known active compound (for example, to produce a mimetic) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled to according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of the ligand and its binding partner are modelled. This can be especially useful where the ligand and/or binding partner change conformation on binding, allowing the model to take account of this in the optimisation of the lead compound.

A template molecule is then selected onto which chemical groups which mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

The activation of DNA repair and DNA damage checkpoint proteins in senescent cells as described herein provides a range of biomarkers which may be used to detect and/or identify senescent cells, for example within a population of dividing cells. The detection of these biomarkers may be useful in the diagnosis of senescence-associated disorders, in the prognosis of individuals with such disorders and the. evalutation of therapies for treating senescence associated disorders.

Another aspect of the invention provides a method of detecting a senescent cell may comprise determining the activation of the DNA damage checkpoint response pathway in said cell.

A cell may be comprised within a sample of cells or tissue obtained from an individual. The presence of an activated DNA damage checkpoint response in a cell of said sample is indicative that the cell is senescent. This may be indicative, for example, that the individual has a senescence associated disorder.

Alternatively, activation of the DNA damage checkpoint response may be determined in a cell in vivo within an individual.

Activation of the DNA damage checkpoint response may be determined as described above. For example, activation may be determined by determining the presence of a DNA damage checkpoint response polypeptide which has been activated by phosphorylation.

The presence of a polypeptide in an active, phosphorylated form may be determined by contacting a sample with an antibody which binds specifically to the phosphorylated polypeptide (i.e. it shows little or no binding to the unphosphorylated polypeptide) and determining the reactivity of the antibody with the sample. Binding of the antibody to one or more cells of the sample is indicative that the one or more cells are in a senescent state.

In some preferred embodiments, the activation of the DNA damage checkpoint response may be determined by determining the presence of one or more senescence associated foci (SAFs) in the nucleus of a cell.

As described above, SAFs may comprise one or more DNA damage checkpoint response polypeptides, including for example γH2AX (C terminally phosphorylated form of Histone H2AX), 53BP1, MDC1, NBS1, RAD50, MRE11, SMC1, and RAD51. The presence of SAFs may be detected by any convenient method, including immunofluorescence, ELISA or immunoblotting.

For example, cells in a sample may be contacted with an antibody which binds to DNA damage checkpoint response polypeptide, and the binding of the antibody to foci of the polypeptide in the nuclei of the cells may be determined. The presence of foci in the nucleus of a cell is indicative that the cell is senescent.

The reactivity of an antibody with a sample may be determined in methods of the invention by any appropriate means. Suitable protocols are well known in the art (see for example Antibodies: A Laboratory Manual E. Harlow and D. Lane, Cold Spring Harbor Laboratory Press, NY, 1988). Tagging with individual reporter molecules is one possibility. The reporter molecules may directly or indirectly generate detectable, and preferably measurable, signals. The linkage of reporter molecules may be directly or indirectly, covalently, e.g. via a peptide bond or non-covalently. Linkage via a peptide bond may be as a result of recombinant expression of a gene fusion encoding antibody and reporter molecule. The actual mode of determining the binding of an antibody molecule is not a feature of the invention and those skilled-in the art are able to choose a suitable mode according to their preference and general knowledge.

Various aspects of the invention relate to the treatment of disorders which are associated with cellular senescence by inhibiting the DNA damage checkpoint response pathway.

An aspect of the invention provides a method of treating a senescence associated disorder in an individual comprising inhibiting the DNA damage checkpoint pathway in said individual.

The DNA damage checkpoint pathway may be inhibited by administering an agent which is a DNA damage checkpoint pathway inhibitor to the individual.

Other aspects of the invention provide a DNA damage checkpoint pathway inhibitor for use in treating a senescence associated disorder and the use of a DNA damage checkpoint pathway inhibitor in the manufacture of a medicament for use in the treatment of a senescence associated disorder.

A DNA damage checkpoint pathway inhibitor may inhibit the kinase activity of one or more of ATM, ATR, CHK1, CHK2 and BRCA1.

Known inhibitors of the DNA damage checkpoint pathway which may be useful in the treatment of senescence associated disorders include ATM inhibitors as described in PCT/GB03/000770 and isomers, salts, solvates, chemically protected forms, and prodrugs thereof. As described above, an ATM inhibitor may have the formula: 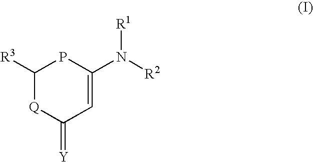
wherein:

one of P and Q is O, and the other of P and Q is CH, where there is a double bond between whichever of Q and P is CH and the carbon atom bearing the R³ group;

Y is either 0 or S;

R¹ and R² are independently hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;

R³ is a phenyl or pyridyl group, attached by a first bridge group selected from —S—, —S(═O)—, —S(═O)₂—, —O—, —NR^{N}— and CR^{C1}R^{C2}— to an optionally substituted C₅₋₂₀ carboaryl group, in which one aromatic ring atom may be replaced by a nitrogen ring atom; the phenyl or pyridyl group and optionally substituted C₅₋₂₀ carboaryl group being optionally further linked by a second bridge group, which is bound adjacent the first bridge group on both groups so as to form an optionally substituted C₅₋₇ ring fused to both the phenyl or pyridyl group and the C₅₋₂₀ carboaryl group, the phenyl or pyridyl group being further optionally substituted;

wherein R^{N} is selected from hydrogen, an ester group, an optionally substituted C₁₋₇ alkyl group, an optionally substituted C₃₋₂₀ heterocyclyl group and an optionally substituted C₅₋₂₀ aryl group;

and R^{C1} and R^{C2} are independently selected from hydrogen, an optionally substituted C₁₋₇ alkyl group, an optionally substituted C₃₋₂₀ heterocyclyl group and an optionally substituted C₅₋₂₀ aryl group.

Other inhibitors may include antibody molecules which bind specifically to one or more polypeptides of the DNA damage checkpoint pathway and reduce or inhibit activity. The production of antibody molecules is described in detail above.

Other inhibitors useful in the treatment of senescence associated disorders may be identified and/or obtained by methods described herein.

Senescence associated disorders include any disorder which is fully or partially mediated by the induction or maintenance of a non-dividing or senescent state in a cell or a population of cells. Examples include coronary disease, impaired wound healing, immune dysfunction, age-related tissue or organ decline, Alzheimer's disease, liver cirrhosis and immuno-senescence caused by chronic infections by agents such as HIV.

A compound which induces cell division in senescent cells as described above may be formulated in a composition. A composition may include, in addition to said compound, a pharmaceutically acceptable excipient, carrier, buffer, stabiliser or one or more other materials well known to those skilled in the art. Such materials should be non-toxic and should not interfere with the efficacy of the active ingredient. The precise nature of the carrier or other material may depend on the route of administration, e.g. oral, intravenous, cutaneous or subcutaneous, nasal, intramuscular, topical or intraperitoneal routes.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at a particular site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

Administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors, and typically takes account of the disorder to be treated, the condition of the individual patient, the site of delivery, the method of administration and other factors known to practitioners. Examples of the techniques and protocols mentioned above can be found in Remington's Pharmaceutical Sciences, 16th edition, Osol, A. (ed), 1980.

The experimental basis for the invention and illustrative embodiments of the invention will now be described in more detail, with reference to the accompanying drawings. All publications mentioned anywhere in the text are incorporated herein by reference.

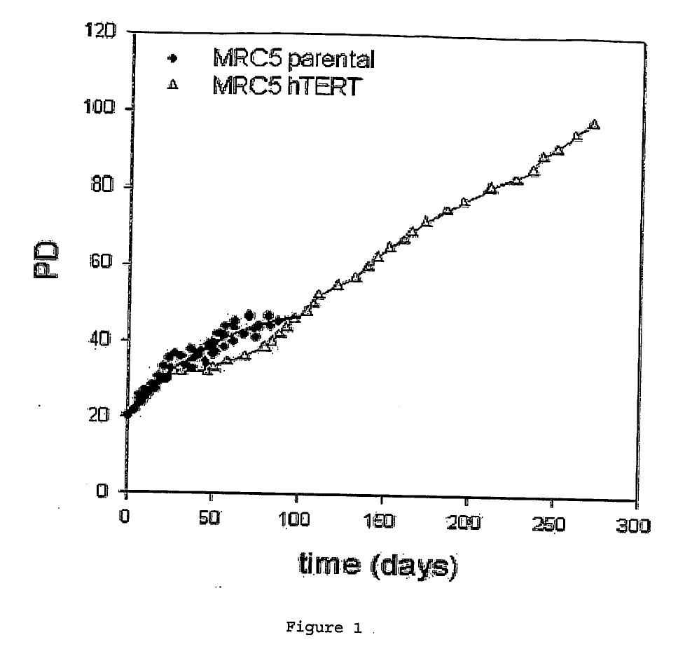

FIG. 1 shows a graph which indicates that hTERT expression in MRC5 cells extends their proliferative capacity in culture.

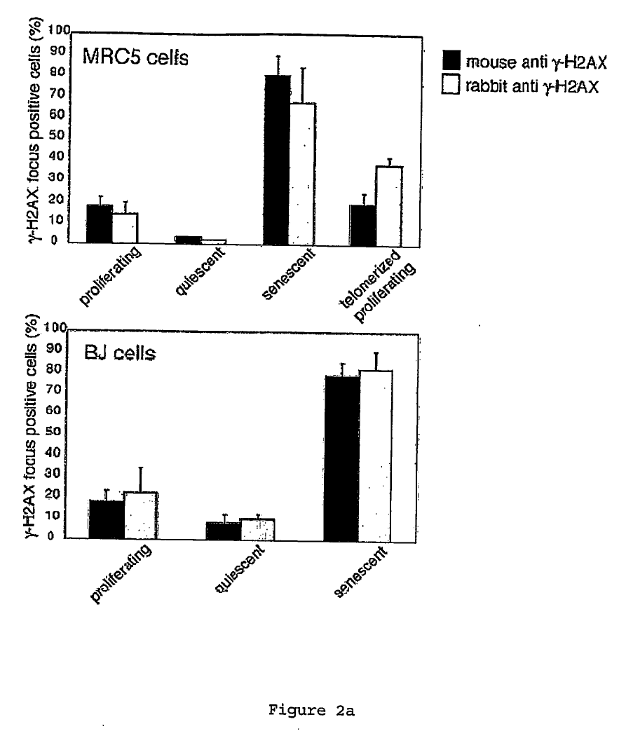

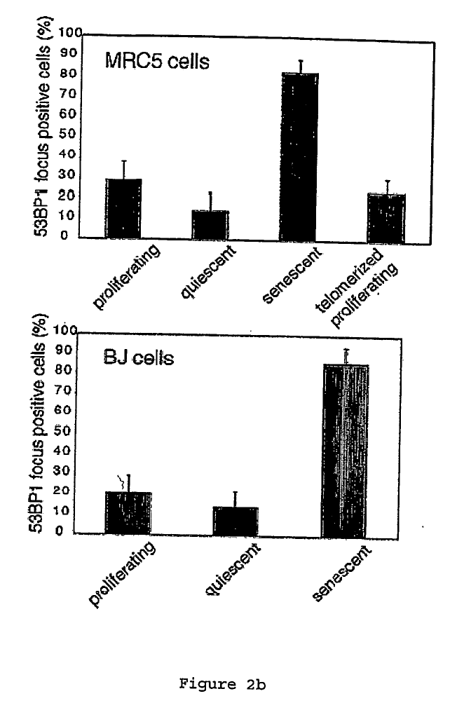

FIGS. 2a and 2b show a quantitation of the fraction of γH2AX or 53BP1 foci positive cells, respectively, in a population. γ-H2AX and 53BP1 foci were detectable by immunofluorescence in senescent cells, and in irRADiated cells but not in quiescent cells. Irradiated cells were exposed to 20Gy of IR and analysed one hour later. Quiescent and senescent cells were not exogenously damaged. γ-H2AX foci were stained with either a mouse monoclonal or an affinity-purified rabbit antibody. Histograms indicate the percentage of cells with at least one clearly detectable focus in young proliferating, quiescent, senescent and telomerized MRC5 cell cultures, and in proliferating and quiescent telomerized (PD>150) and senescent (PD˜75) BJ cell cultures.

The absolute numbers of MRC5 γ-H2AX positive cells were as follows: proliferating cells—30 cells out of 168 and 60/138 when using the monoclonal or the polyclonal anti-γ-H2AX reagent respectively; quiescent cells—6/183 and 4/200 respectively; senescent cells—213/265 and 163/236; proliferating telomerized cells—24/121 and 34/182. The absolute numbers of BJ γ-H2AX positive cells were; proliferating cells—53/292 and 50/224; quiescent cells—17/211 and 14/142; senescent cells—164/208. The numbers of MRC5 53BP1 foci positive cells were: proliferating—57/196, quiescent—25/174, senescent—64/77, telomerized—42/182; the numbers of BJ 53BP1 foci positive cells were; proliferating—53/266, quiescent—28/198, senescent—166/193. In total, 4457 cells were screened in this analysis.

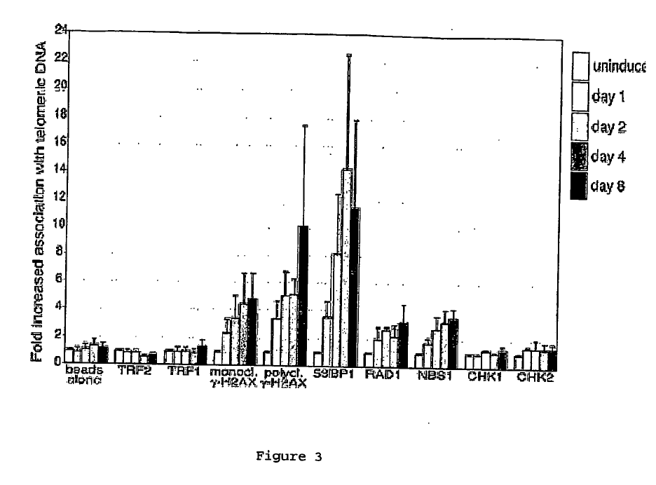

FIG. 3 shows that uncapped telomeres associate with markers of the DNA damage checkpoint response. Formaldehyde crosslinked chromatin was prepared from T19 cells expressing ΔTRF2 for up to 8 days. Chromatin immunoprecipitations were performed with the indicated antibodies. The Y axis value represents the calculated fold increase of the association of the indicated antigen with telomeric DNA upon induction of ΔTRF2.

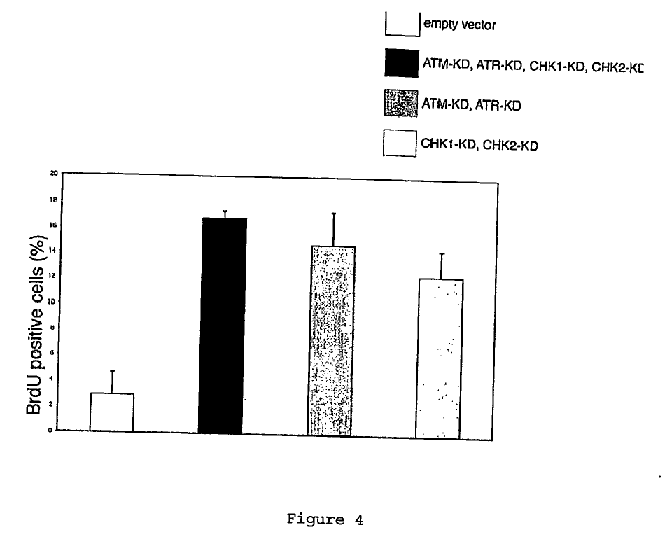

FIG. 4 shows that checkpoint inactivation in senescent cells induces cell cycle progression and DNA synthesis. Percentage of senescent BJ cells microinjected with plasmids expressing dominant negative alleles of the indicated DNA damage checkpoint proteins is shown.

Methods

Cell culture

MRC5 and BJ HDFs were grown under standard tissue culture conditions in DMEM supplemented with 10% foetal calf serum. MRC5 and BJ cells were from ATCC and telomerized BJ cells were described in Bodnar, A. G. et al. Science 279, 349-352 (1998). Senescence of cultures was evaluated by: failure to reach confluence after three weeks in culture from the last 1:2 passage, failure of more than 95% of cells to incorporate BrdU after a 24-hours feeding period, widespread expression of senescence-associated β-galactosidase, morphological features and increased number of PML bodies. The human cell line T19, a kind gift of T. de Lange, expressing an inducible dominant-negative allele of TRF2 was maintained as described in van Steensel, B. et al Cell 92, 401-13. (1998). Typically, cells were irradiated with 20Gy and analysed 1 hour later.

Immunofluorescence Microscopy.

Cells were grown on poly-L-lysine (Sigma) coated glass coverslips. Fixation was either with 2% paraformaldehyde in PBS for 10 min, after which cells were permeabilized with 0.5% NP-40 for 10 min, or with a 50% methanol 50% acetone mixture for 2 min at room temperature. Coverslips were blocked in PBG (0.2% cold water fish gelatine, 0.5% BSA in PBS) and incubated with primary antibody in PBG. Cells were then washed with PBG and incubated with fluorescein isothiocyanate (FITC)-conjugated or rhodamine-conjugated secondary antibodies (Jackson Laboratories) in the presence of the DNA dye TOTO-3 (Molecular Probes). Coverslips were finally washed with PBG and PBS and mounted. Confocal sections were obtained with a Bio-rad confocal laser microscope by sequential scanning. Primary antibodies used in immunofluorescence studies were: mouse and rabbit anti-γ-H2AX (Upstate Biotechnology), anti-53BP1, anti-pS/TQ (Cell Signaling Technology), anti-NBS1 (Oncogene), anti-SMClpS966 (Bethyl Laboratories), anti-MDC1 (Goldberg, M. et al. Nature 421, 952-6 (2003)), anti-PML (Santa Cruz Biotechnology). Comparative immunofluorescence analysis of foci in proliferating, quiescent, senescent and telomerized cells was always carried out in parallel.

Immunoblotting

Cell extracts were prepared by lysis in TEB150 buffer (50 mM Hepes pH 7.4, 150 mM NaCl, 2 mM MgCl₂, 5 mM EGTA pH 8, 1 mM DTT, 0.5% Triton X-100, 10% glycerol, 1 mM Na₃VO₄, 1 μM microcystin-LR, 1x Complete EDTA-free Protease Inhibitor Cocktail (Roche)) or lysed directly in SDS-PAGE sample buffer. Proteins were resolved by SDS-PAGE, transferred to nitrocellulose and probed with the appropriate antibody. Rabbit anti-RAD17 (H-300), rabbit anti-H2A (H-124), mouse monoclonal anti-CHK1 (G-4), and rabbit anti-p21 (C19) antibodies were purchased from Santa Cruz Biotechnology. Anti-CHK1 pS345, anti-CHK2 pT68 and anti-p53 pS15 rabbit antibodies were purchased from Cell Signaling Technology. Anti-SMC1 pS966 and anti-SMC1 rabbit antibodies were purchased from Bethyl Laboratories. Mouse monoclonal anti-FLAG (M2) antibodies were purchased from Sigma; mouse monoclonal anti-γ-H2AX antibodies were purchased from Upstate Biotechnology and rabbit anti-CHK2 antibodies were purchased from Abcam. Results shown are representative of at least three experiments.

Chromatin Immunoprecipitation (ChIP)

ChIPs were carried out as described in d'Adda di Fagagna, F. et al. Curr Biol 11, 1192-6 (2001) with the exception that cesium chloride gradient purification was omitted and cell lysis and immunoprecipitations were carried out in the presence of 1 μM microcystin-LR. Histogram bar heights represent the mean value of at least ten immunoprecipitations per antigen. Anti-TRF1 and TRF2 (N-20) antibodies were from Santa Cruz Biotechnology, mouse and rabbit anti-γ-H2AX from Upstate Biotechnology, anti-53BP1 were a gift from T. Halazonetis, anti-RAD1 as described in Freire, R. et al. Genes Dev 12, 2560-73 (1998), anti-NBS1 from Oncogene and as in Goldberg, M. et al. Nature 421, 952-6 (2003), anti-Ku and DNA-PKcs as in d'Adda di Fagagna, (2001) supra, anti-CHK1 from Upstate, anti-CHK2 from Oncogene and anti-CHK2 T68 from Cell Signaling Technology.

Microinjection

Cells were plated on a poly-L-lysine coated glass dish (Bioptechs, PA), incubated in Nut Mix F-12 (Ham) medium (Gibco) plus 10% newborn calf serum. 100 ng/μl of each plasmid and pH2B-YFP as an injection marker were microinjected in senescent cell nuclei using Fentotips (Eppendorf). BrdU incorporation was monitored by Amersham Cell Proliferation Kit.

All microinjections experiments were performed by expression of the different combinations of plasmids in parallel in different cells on the same dish. Values reported are the results of more than three separate experiments, using two different batches of senescent cells.

The DNA-Damage Response is Triggered in Senescent HDFs

MRC5 cells are human diploid fibroblasts from a normal healthy donor which usually proliferate for around 45 population doublings (PD) before reaching senescence. hTERT was expressed in these cells by retroviral transduction and the proliferative capacity of transduced cells monitored relative to parental cultures.

The hTERT-transduced MRC5 cell population grew vigorously, well beyond the natural proliferative limit of this cell line (FIG. 1). Telomere restriction fragment analysis revealed that telomeres were maintained at a stable size in the hTERT transduced but not the parental cultures. It can therefore be concluded that MRC5 cells undergo cellular senescence because of telomere attrition. MRC5 and BJ cells—another HDF line that undergoes senescence triggered by telomere shortening (Bodnar, 1998 supra)—were used to study the relationship between telomere-initiated senescence and the DNA-damage response.

Antisera against the phosphorylated form of H2AX (γ-H2AX) and against 53BP1 were used in immunofluorescence experiments with senescent MRC5 cells and with control cultures of X-irradiated cells and contact-inhibited non-dividing quiescent cells. Virtually every cell in the X-irradiated population possessed a large number of γ-H2AX and 53BP1 nuclear foci. Moreover, although very few undamaged quiescent cells displayed foci, the vast majority of cells in the senescent MRC5 cell population had a small number of clearly detectable γ-H2AX and 53BP1 foci. Dual-staining studies indicated that, as in irradiated cells, the foci of γ-H2AX and 53BP1 in senescent cells were largely coincident (FIG. 2).

Blindly chosen fields of cells were quantitatively examined for γ-H2AX and 53BP1 focus formation. When young (PD<30) proliferating cultures were probed with either a mouse monoclonal or an affinity-purified rabbit polyclonal antibody raised against γ-H2AX, 17% and 15% of the cells, respectively, had at least one detectable γ-H2AX focus. These foci presumably represent DSBs arising during DNA replication because only 3% (monoclonal γ-H2AX antibody) and 2% (polyclonal γ-H2AX antibody) of cells in quiescent populations possessed such foci. In striking contrast, between 70% and 80% of cells in senescent (PD˜45) MRC5 cultures contained γ-H2AX foci. Furthermore, consistent with the fact that the proportion of senescent cells progressively increases in cultures of increasing PDs, the proportion of γ-H2AX foci positive cells became progressively augmented as cultures approached senescence. Importantly, only 18-20% of proliferating telomerized cells (PD>70) possessed γ-H2AX foci, which is very similar to the values obtained with non-telomerized young proliferating cells. As the telomerized cells had been grown in culture for significantly more PDs than the senescent cells, this indicates that prevalence of cells with γ-H2AX foci does not reflect prolonged time in culture per se. Instead, the prevalence of cells with γ-H2AX is specifically associated with MRC5 cell senescence. Consistent with this being a general feature of fibroblasts undergoing telomere-initiated senescence, around 80% (79% for monoclonal γ-H2AX antibody; 82% for polyclonal γ-H2AX antibody) of senescent BJ cells also possessed γ-H2AX foci, whereas such foci were detected in only around 20% (18% and 22%) of proliferating BJ cells, and in less than 10% (8% and 9.8%) of quiescent BJ cells.

Very similar results were obtained when the quantitative analysis was extended to 53BP1. Thus, 83% of cells in senescent MRC5 populations had at least one clearly distinguishable 53BP1 focus, whereas the corresponding values for proliferating, quiescent and telomerized MRC5 cells were 29%, 14% and 23%, respectively. Likewise, 86% of senescent BJ cells displayed 53BP1 foci, while the figures for proliferating and quiescent BJ cells were 20% and 14%, respectively. These data reveal that widespread formation of γ-H2AX and 53BP1 foci is associated with telomere-initiated senescent for the two different HDF cell lines tested, and provide indication that it may be a general feature of HDF—and possibly other cell types—undergoing telomere-initiated senescence. The formation of such foci may thus be useful as a biomarker for telomere-initiated senescence.

The above results provide indication that ATM and ATR are activated in senescent HDFs. In line with this, prevalent focal staining of senescent cells was also detected by an antiserum raised against the ATM/ATR phosphorylation consensus sequence (phospho-Ser/Thr-Gln). Moreover, □-H2AX foci in senescent populations colocalized with foci of the DNA-damage checkpoint protein MDC1, the checkpoint and DSB repair factor NBS1, and the Ser-933 phosphorylated form of SMC1. In contrast, PML bodies—sub-nuclear structures that to some extent colocalize with sites of DNA damage at late time points (Carbone, R. et al Oncogene 21, 1633-1640 (2002))—colocalized only poorly with γ-H2AX foci, despite them being present in increased numbers in senescent cells. Taken together, these data reveal that ATM/ATR dependent phosphorylation of γ-H2AX and foci formation by a range of DNA repair and DNA-damage signalling proteins takes place in senescent HDFs. These subnuclear structures are termed ‘SAFs’ (senescence-associated foci) herein.

As anticipated from the immunofluorescence studies, immunoblotting with antibodies directed against γ-H2AX and SMC1 phosphoylated on Ser-966 confirmed the accumulation of these epitopes in senescent cells. More importantly, immunoblotting allowed the analysis to be extended to DNA-damage response markers that were less amenable to immunofluorescence approaches.

ATM/ATR-dependent phosphorylation of RAD17 on Ser-645 is triggered by DNA damage and appears to be necessary for full checkpoint activation. Undamaged proliferating early PD or late PD telomerized cells displayed significant RAD17 Ser-645 phosphorylation, reflecting the involvement of RAD17 and ATR in monitoring normal S-phase progression. However, while RAD17 Ser-645 phosphorylation was almost undetectable in quiescent cells, it was clearly evident in extracts from senescent cells. RAD17 is therefore phosphorylated and activated in senescent HDFs.

Full execution of DNA-damage-induced cell cycle arrest requires ATM and/or ATR mediated phosphorylation of CHK1 and CHK2 on Ser-345 and Thr-68, respectively, resulting in CHK1 and CHK2 activation and phosphorylation of their downstream targets. Antisera against the CHK1 and CHK2 phosphorylation sites were used to monitor their status in senescent cells. Because CHK1 is down-regulated at both the transcriptional and protein levels in non-proliferating cells, the amounts of this protein were normalised before analysis. Both CHK1 and CHK2 were found to be phosphorylated on these key activating residues in senescent cells but not in proliferating young or telomerized HDFs, or in quiescent cells.

DNA-Damage Markers Associate with Uncapped Telomeres

The endogenous telomere capping factor TRF2 may be stripped off telomeres by expression of a dominant-negative truncated form of the protein (□TRF2). The ensuing synchronous uncapping of telomeres triggers bona fide senescence in human fibroblasts—and ATM and p53 dependent apoptosis in other cell types—without the formation of dicentric chromosomes and subsequent chromosomal breakage (Karlseder, J. et al Science 295, 2446-9 (2002)). Senescence can therefore be induced in such a system while normal telomere length is preserved (van Steensel, 1998 supra).

A human immortalised fibroblast cell line containing an stably-integrated inducible ΔTRF2 expression construct was grown under inducing conditions for various lengths of time. Extracts from these cells were then examined by immunoblotting, together with control extracts from uninduced cells or irradiated cells. Strikingly, expression of FLAG epitope-tagged ΔTRF2 led to the specific and robust accumulation of γ-H2AX, Ser-645 phosphorylated RAD17 and Ser-966 phosphorylated SMC1. The eventual degree of induction of these markers by ΔTRF2 was similar to that achieved by 20Gy of IR. Consistent with these results, immunofluorescence analysis revealed the widespread induction of nuclear foci for 53BP1, MCD1 and γ-H2AX in the ΔTRF2-induced cell population. At the latest time points tested, the activated phosphorylated forms of CHK1 and CHK2 were also clearly detectable in extracts from the induced cells. Taken together, these data reveal that the ΔTRF2-induced fibroblast senescence system triggers the DNA-damage response, and therefore accurately mimics the induction of these events in naturally senescing HDF cultures.

Chromatin immunoprecipitation (ChIP) was used to test for the possible in vivo recruitment of DNA repair and checkpoint factors to telomeric DNA. Uninduced and induced cells were treated with the cross-linking agent formaldehyde and performed immunoprecipitations with a panel of antibodies attached to sepharose beads. The recovered DNA was then subjected to dot-blot DNA hybridisation with a telomeric probe and the ratio of telomeric DNA immunoprecipitated from induced as opposed to uninduced cells was calculated. After normalising the resulting data by the corresponding ratio obtained from parallel measurements of hybridisation to Alu repeat sequences, the fold change in association of proteins to telomeric regions after ΔTRF2 induction was calculated.

While the low amount of telomeric (or Alu) DNA non-specifically immunoprecipitated by beads alone did not change significantly throughout the experiment, the induction of ΔTRF2 reduced the association of endogenous TRF2 with telomeric DNA by almost 50% (FIG. 3). However, the association of TRF1—a different telomere binding protein—was essentially unchanged by ΔTRF2 induction, providing a control for the integrity of telomeric DNA. In striking contrast, ΔTRF2-induction led to substantial increases in the amount of telomeric DNA retrieved by antisera against γ-H2AX (monoclonal and polyclonal) or 53BP1 (FIG. 3). In addition, ΔTRF2-induction produced significant and reproducible increases in telomeric association of NBS1 and of the DNA-damage checkpoint protein RADl. This latter result is consistent with our observation of RAD17 phosphorylation in senescenct cells. Significantly, however, although immunoblotting clearly revealed CHK1 and CHK2 phosphorylation upon ΔTRF2 induction, no enhancement of telomere binding by these factors or their active phospho-forms was observed, even at late time-points (FIG. 3). These data are in line with recent work demonstrating that the phosphorylated, active forms of these kinases do not intimately associate with sites of DNA-damage but instead distribute throughout the nuclear volume (Lukas, C. et al. Nat Cell Biol (2003)). Taken together, these results show that telomere de-protection leads to the accumulation of a range of DNA-damage response proteins on telomeric DNA and that this leads to induction of a bona fide DNA-damage response.

Inhibition of DNA-Damage Kinases Restarts Cell Cycle Progression in Senescent Cells

To see whether the maintenance of senescence requires the continual functioning of the DNA-damage response pathway, senescent BJ cells were micro-injected with combinations of plasmids expressing dominant negative kinase dead (KD) forms of DNA-damage response kinases (ATM-KD, ATR-KD, CHK1-KD and CHK2-KD), or with an equal amount of the parental vector (in parallel and on the same tissue-culture dish). The cells were then monitored for DNA replication by measuring BrdU incorporation over a 3.5-day period (FIG. 4). As expected from a senescent cell population, only 3% (11 /348) of cells microinjected with the empty vector performed at least one round of DNA synthesis. By contrast, 16% (25/152) of cells microinjected with the four KD constructs incorporated BrdU. Furthermore, expression of pairwise conbinations of ATM-KD and ATR-KD, or CHK1-KD and CHK2-KD, induced BrdU incorporation in 15(27/183) and 12% (19/160) of microinjected cells, respectively. The incorporation of BrdU observed was indeed due to genuine S-phase progression, as it was accompanied by expression of MCM5, a factor that is a highly specific marker for chromosomal DNA replication but not DNA-repair. These data therefore reveal that inhibition of DNA damage checkpoint proteins can restart cell cycle progression in senescent cells.

In summary, human fibroblasts undergoing telomere-initiated senescence display features that are qualitatively indistinguishable from those elicited by radiation-induced DNA DSBs. These features include activation of the DNA-damage transducer kinases ATM and ATR, accumulation of foci of γ-H2AX, 53BP1, MDC1, NBS1 and the Ser-933 phosphorylated form of SMC1 and activation of the downstream effector kinases CHK1 and CHK2. In addition, these responses are also engaged in a model system where human cell senescence is triggered by telomere deprotection. DNA-damage response proteins were physically targeted to telomeric DNA in this system. Interfering with the DNA-damage response allows a proportion of senescent cells to resume DNA synthesis. These observations provide the basis for a mechanistic understanding of telomere-initiated senescence in human somatic cells. Specifically, they provide indication that the senescence programme of HDFs is triggered by a DNA-damage response elicited by critically shortened telomeres.

The results set out herein provide indication that senescence is an actively maintained condition. Foci were reproducibly detected up to more than two months after cells had entered a senescent state. Therefore, the phosphorylation of these foci must be actively sustained. Furthermore, the ability to recover S-phase progression by inactivation of a specific set of checkpoint proteins provides strong indication that senescence can only be maintained if these proteins are continuously active.

The discovery that senescence is associated with a set of DNA damage markers could allow for both the development of therapies for senescence associated disorders and the identification of senescent cells in normal and pathological tissues and help to understand their role in complex processes such as tumour development and ageing.

1. A method of identifying an agent for the treatment of a senescence associated disorder comprising:
contacting a test compound with a DNA damage checkpoint response polypeptide;
determining binding of the polypeptide by the test compound,
binding of the DNA damage checkpoint pathway polypeptide being indicative that the test compound is a candidate agent for the treatment of senescence associated disorders.

2. A method according to claim 1 comprising determining the activity of the polypeptide in the presence and absence of said test compound.

3. A method according to claim 1 wherein the polypeptide is selected from the group consisting of ATM, ATR, ATRIP, CHK1, CHK2, BRCA1, NBS1, RAD50, MRE11, CDC25C, 14-3-3σ, CDK2/cyclin E, CDK2/cyclin B1 53BP1, MDC1, histone variant γH2AX, RAD17, RAD1, RAD9, HUS1 and MRC1.

4. A method according to any one of the preceding claims wherein activity is determined by determining the phosphorylation of said polypeptide.

5. A method according to claim 4 wherein the polypeptide is selected from the group consisting of ATRIP, CHK1, CHK2, BRCA1, NBS1, RAD50, MRE11, CDC25C, 14-3-3α, CDK2/cyclin E, CDK2/cyclin B1 53BP1, MDC1, histone variant γH2AX, SMC1, RAD17, RAD1, RAD9, HUS1 and MRC1.

6. A method according to claim 1 wherein activity is determined by the determining the kinase activity of said polypeptide.

7. A method according to claim 6 wherein the polypeptide is selected from the group consisting of ATM, ATR, Chk1 or Chk2.

8. A method according to claim 1 wherein the senescence related disorder is coronary disease, impaired wound healing, immune dysfunction, age-related tissue or organ decline, Alzheimer's disease, liver cirrhosis or immuno-senescence caused by chronic infection.

9. A method of screening for an agent for the treatment of a senescence associated disorder, which comprises:
providing a DNA damage checkpoint pathway;
exposing the pathway to a test compound under conditions which would normally lead to the activation of the DNA repair pathway; and
determining the activation of the ATM/ATR DNA damage signalling pathway in the presence relative to the absence of test compound.

10. A method according to claim 9 wherein said pathway is comprised in a eukaryotic cell.

11. A method according to claim 10 wherein the cell is a mammalian cell.

12. A method according to claim 9 wherein activity is determined by the determining the phosphorylation of a DNA damage checkpoint response polypeptide.

13. A method according to claim 12 wherein the polypeptide is selected from the group consisting of ATM, ATR, ATRIP, CHK1, CHK2, BRCA1, NBS1, RAD50, MRE11, CDC25C, 14-3-3α, CDK2/cyclin E, CDK2/cyclin B1 53BP1, MDC1, histone variant γH2AX, SMC1, RAD17, RAD1, RAD9, HUS1 and MRC1.

14. A method according to claim 9 wherein activity is determined by determining the activity of a DNA damage checkpoint kinase.

15. A method according to claim 14 wherein the a DNA damage checkpoint kinase is selected from the group consisting of ATM, ATR, Chk1 or Chk2.

16. A method according to claim 9 wherein activation is determined by determining the presence of nuclear foci of a polypeptide selected from the group consisting of γH2AX, 53BP1, MDC1, NBS1/RAD50/MRE11, SMC1 and RAD51.

17. A method according to claim 1 comprising determining the ability of said test compound to induce cell cycle progression in a senescent cell.

18. A method according to claim 17 comprising identifying said test compound as an agent which induces cell cycle progression in a senescent cell.

19. A method according to claim 18 comprising isolating said test compound.

20. A method according to claim 19 comprising formulating said test compound in a pharmaceutical composition with a pharmaceutically acceptable excipient, vehicle or carrier.

21. An agent obtained by a method of claim 1.

22. A method of producing a pharmaceutical composition for use in the treatment of a senescence associated disorder comprising;
identifying a compound which induces cell cycle progression in a senescent cell using a method according to claim 1; and,
admixing the compound identified thereby with a pharmaceutically acceptable carrier.

23. A method according to claim 22 comprising the step of modifying the compound to optimise the pharmaceutical properties thereof.

24. A method for preparing a pharmaceutical composition for treating a senescence associated disorder
comprising;
identifying an agonist/antagonist of the DNA damage checkpoint response,
synthesising the identified compound, and;
incorporating the compound into a pharmaceutical composition.

25. A method of identifying a senescent cell in a sample comprising,
providing a sample comprising one or more cells, and;
determining the activation of the DNA damage checkpoint response pathway in said one or more cells.

26. A method according to claim 25 wherein the presence of an activated DNA damage checkpoint response pathway in a cell of said sample is indicative that the cell is senescent.

27. A method according to claim 25 wherein activation is determined by determining the kinase activity of ATM, ATR, CHK1 or CHK2.

28. A method according to claim 25 wherein activation is determined by determining the phosphorylation of ATM, ATR, ATRIP, CHK1, CHK2, BRCA1, NBS1, RAD50, MRE11, CDC25C, 14-3-3α, CDK2/cyclin E, CDK2/cyclin B1 53BP1, MDC1, histone variant γH2AX, SMC1, RAD17, RAD1, RAD9, HUS1 and MRC1.

29. A method according to claim 25 wherein activation is determined by determining the presence of nuclear foci of a polypeptide selected from the group consisting of γH2AX, p53BP1, MDC1, NBS1, RAD50, MRE11, SMC1, and RAD51.

30. A method of treating a senescence related disorder in an individual comprising inhibiting the ATM/ATR DNA damage checkpoint pathway in said individual.

31. A method according to claim 30 wherein the senescence related disorder is coronary disease, impaired wound healing, immune dysfunction, age-related tissue or organ decline, Alzheimer's disease, liver cirrhosis or immuno-senescence caused by chronic infection.

32. A method according to claim 30 comprising administering an DNA damage checkpoint pathway inhibitor to said individual.

33. A method according to claim 32 wherein the DNA damage checkpoint pathway inhibitor is an inhibitor of the kinase activity of one or more of ATM, ATR, CHK1, CHK2 and BRCA1.

34. A method according to claim 30 wherein the inhibitor is obtained by a method comprising:
contacting a test compound with a DNA damage checkpoint response polypeptide;
determining binding of the polypeptide by the test compound,
binding of the DNA damage checkpoint pathway polypeptide being indicative that the test compound is a candidate agent for the treatment of senescence associated disorders.

35. A method according to claim 30 wherein the inhibitor has the formula: 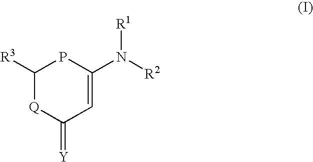
or is an isomer, salt, solvate, chemically protected form, or prodrug thereof, wherein:
one of P and Q is O, and the other of P and Q is CH, where there is a double bond between whichever of Q and P is CH and the carbon atom bearing the R³ group;
Y is either 0 or S;
R¹ and R² are independently hydrogen, an optionally substituted C₁₋₇ alkyl group, C₃₋₂₀ heterocyclyl group, or C₅₋₂₀ aryl group, or may together form, along with the nitrogen atom to which they are attached, an optionally substituted heterocyclic ring having from 4 to 8 ring atoms;
R³ is a phenyl or pyridyl group, attached by a first bridge group selected from —S—, —S(═O)—, —S(═O)₂—, —O—, —NRN— and CR^{C1}R^{C2}—to an optionally substituted C₅₋₂₀ carboaryl group, in which one aromatic ring atom may be replaced by a nitrogen ring atom;
the phenyl or pyridyl group and optionally substituted C₅₋₂₀ carboaryl group being optionally further linked by a second bridge group, which is bound adjacent the first bridge group on both groups so as to form an optionally substituted C₅₋₇ ring fused to both the phenyl or pyridyl group and the C₅₋₂₀ carboaryl group, the phenyl or pyridyl group being further optionally substituted;
wherein R^{N} is selected from hydrogen, an ester group, an optionally substituted C₁₋₇ alkyl group, an optionally substituted C₃₋₂₀ heterocyclyl group and an optionally substituted C₅₋₂₀ aryl group;
and R^{C1} and R^{C2} are independently selected from hydrogen, an optionally substituted C₁₋₇ alkyl group, an optionally substituted C₃₋₂₀ heterocyclyl group and an optionally substituted C₅₋₂₀ aryl group.

36-40. (canceled)
